(19) United States
(12) Patent Application Publication
(10) Pub. No.: US 2017/0258990 A1
(43) Pub. Date: Sep. 14, 2017

(54) PROTECTIVE COVER TYPE SAFETY DEVICE FOR SYRINGES

(71) Applicant: Ching Hsiang Wei, Taichung City (TW)

(72) Inventor: Ching Hsiang Wei, Taichung City (TW)

(21) Appl. No.: 15/065,869

(22) Filed: Mar. 10, 2016

(51) Int. Cl. A61M 5/162; A61M 5/32

(52) U.S. Cl. CPC A61M 5/1626; A61M 5/3202; A61M 5/3216; A61M 2005/3217

(57) ABSTRACT

A protective cover type safety device for syringes includes a first buckle seat, a second buckle seat which can be engaged with the first buckle seat, and a protective cover which is pivotably connected with the first buckle seat. The first buckle seat and the second buckle seats are clamped and positioned to a needle seat of a syringe, and the protective cover is able to swing between a first position and a second position, and limited at a third position. The protective cover at the third position covers a needle body of the syringe to prevent the needle body from being exposed or piercing or hurting people.

### FIELD OF THE INVENTION

The present invention relates to safety of syringes, and particularly relates to a protective cover type safety device placed over used syringes for protection purpose, by means of a one-finger motion made by the user.

### DESCRIPTION OF THE RELATED ART

For safety concerns, syringes are required to be covered/capped by safety devices in order to prevent the needle body from contacting or hurting other people. The problems for the design and production of safety devices include that the shape, structure, and actuation patterns have to be individually made according to the individual syringe types. Currently, there are no known “universal” syringe safety devices, namely, those that can be used on two or more different safety devices. The shapes and structures of the current safety devices are excessively complex, so that the production cost is high and it is hard to be mass-produced. Safety devices increase the volume and weight of the syringes such that they can be inconvenient for medical personnel may to handle. Moreover, waste syringe recycling costs are calculated based on the volume and weight of syringes, therefore, the safety devices that are overweight or oversized increase the waste syringe recycling costs, which have become a financial burden for medical treatment institutions. Besides, the need to change the original shape of needle seats of syringes to fit with the safety devices makes the production more difficult.

### SUMMARY OF THE INVENTION

The purpose of the present invention is to provide a protective cover type safety device for solving the difficulties and problems mentioned above.

In order to achieve the above said purpose, a protective cover type safety device for syringes comprises a first buckle seat, a second buckle seat and a protective cover.

The first buckle seat includes a positioning portion and a wing coupled to each of two sides of the positioning portion, and each of the two wings has a fastener. The exterior of the positioning portion of the first buckle seat has a pivoting portion.

The second buckle seat includes a positioning portion and a wing coupled to each of two sides of the positioning portion, and each wing of the second buckle seat has a fastener. A limiting member extends from the top of each wing of the second buckle seat, and the fastener of the second buckle seat is connected with the fastener of the first buckle seat to clamp and position the first buckle seat and the second buckle seat to the exterior of the needle seat of a syringe.

The protective cover includes a back plate, two side plates respectively coupled to both sides of the back plate, a top plate coupled to the top of the back plate and the top of the two side plates. The back plate, the side plates and the top plate form a first accommodating space and a second accommodating space of the protective cover. The first accommodating space communicates with the second accommodating space. The bottom edge of the back plate has a round shaft, two extension arms are formed on the two side plates and extend toward the left and right sides. A hugging arm extends from an end of each of the two extension arms, and the two hugging arms are parallel to each other.

The protective cover is pivotably connected to the first buckle seat by the round shaft and can swing reciprocally between a first position and a second position about the round shaft. The protective cover is pushed from the first position and latched to a third position by an external push, and thus unable to return to the first position.

The protective cover at the second position embraces the needle cover of the syringe by the two hugging arms. The protective cover swings outwardly from the second position to the first position. The protective cover is offset from the needle cover of the syringe, so as to remove the needle cover and the needle body is ready for injection. After the needle body is used, an external pushing force is applied to the protective cover. The protective cover swings from the first position to the third position, and the protective cover of the third position is latched to the bottom of the limiting members by passing the two hugging arms through the limiting members of the second buckle seat. The protective cover accommodates the needle body and needle seat of the syringe by the first accommodating space and the second accommodating space.

In the embodiment, the fastener of the first buckle seat is a square fastener with a square hole and the fastener of the second buckle seat is a protruding member which is combined with the fastener.

In the embodiment, a flange extends from an upper end of the positioning portion of the first buckle seat. The needle seat has a platform with an expanded diameter and located at a position above the first buckle seat and the second buckle seat, and the flange is stopped at the platform.

In the embodiment, a flange extends from a lower end of the positioning portion of the first buckle seat. A plurality of ribs are formed on an outer wall of the needle seat and parallel to an axial direction of the needle seat. The ribs extend from the bottom of the needle seat, so that a plurality of outwardly protruding latching portions are formed at the bottom edge of the needle seat. The flange at the lower end of the first buckle seat is stopped at the latching portions.

In the embodiment, the flanges at upper and lower ends of the positioning portion of the first buckle seat have an arc end surface.

In the embodiment, an anti-slip structure is disposed on opposite surfaces of the positioning portions of the first buckle seat and the second buckle seat.

In the embodiment, the protective cover has a bevel face disposed at a bottom of the two hugging arms. The second buckle seat has a guiding portion formed at a top of the limiting members for guiding the bevel faces to move downward, the hugging arms are guided below the limiting members, so that the protective cover enters into the third position. The bottom of the limiting members has a stop portion, the hugging arms are pushed below the limiting members and stopped by the stop portions. The hugging arms cannot be separated in an opposite direction.

In the embodiment, the two side plates of the protective cover have protruding clamping ribs disposed on two opposing inner surfaces of the second accommodating space.

In the embodiment, the back plate of the protective cover has a pushing surface.

In the embodiment, the positioning portions of the first buckle seat and the second buckle seat have opposite surfaces in the same geometric shape as that of the cross-section of the needle seat.

The function of the present invention:

By means of clamping a needle seat of a syringe between a first buckle seat and a second buckle seat that are combined face to face, the present invention ensures easy installation on a syringe.

By means of a protective cover that can swing, the position of the protective cover can be changed according to different situations. When the protective cover is in the second position, it can grasp the needle cover of an unused syringe so that the needle cover wouldn't swing. The protective cover can be swung from the second position to the first position, thus allowing the needle cover to be removed so that the needle body can be used for injection. When the protective cover is in the first position, it does not hinder the injection operation. When the protective cover is moved from the first position to the third position, it covers the used need body so as to prevent the needle body from hurting people. Moreover, the protective cover at the third position can not return back to the first position or the second position, so that after use of the needle body, the needle body will not be exposed.

The first buckle seat and the second buckle seat are connected to each other by the two fasteners so as to be tightly fit to the needle seat of the syringe. Since the fasteners are connected to each other and difficult to be separated from each other, the first buckle seat and the second buckle seat are not easily separated from the needle seat so as to ensure that the safety device of the present invention is not separated from the needle seat.

By means of the flanges at the top end and the bottom end of the first buckle seat, the safety device of the present invention is further secured at the platform and the latching portion of any known type of the intravenous catheter, so that the safety device of the present invention does not move along the axis of the needle seat of the syringe and thus provide more stability.

The arc end surfaces of the flanges avoid the flanges from interfering with the needle seat of the syringe, so that the flanges can easily be restricted at the platform and below the latching portion.

By means of the anti-slip structures disposed at the opposite surfaces of the positioning portions of the first buckle seat and the second buckle seat, the friction and therefore the stability are enhanced when the first buckle seat and the second buckle seat are connected with the needle seat of the syringe so as to prevent sliding movement relative to each other.

The bevel faces at the bottoms of the two hugging arms of the protective cover as well as the guiding portions of the second buckle seat enable the protective cover to move from a first position or a second position to a third position. By means of the restriction provided by the stop portions of the second buckle seat and the two hugging arms, the protective cover is restricted at the third position, and cannot return to the first position or the second position anymore.

The clamping ribs at the two side plates of the protective cover clamp the needle body in the second accommodating space.

The pushing surface of the back plate of the protective cover allows a user to push the protective cover from the first position until the third position with only one finger.

The opposite surfaces of the positioning portions of the first buckle seat and the second buckle seat have the geometrical shapes that are identical to the cross section of the needle seat, so that the first buckle seat and the second buckle seat can be installed on syringes with different shapes, so that changing the original shape of needle seats of syringes would not be necessary.

The safety device of the present invention can be produced independently, and its production line is not combined with the production line of syringes, so that the problems encountered during alteration of the production lines of syringes and the costs of solving these problems would be avoided.

The present invention can be used with any known types of syringes such as intravenous catheter, butterfly syringes, attached syringes etc.

### BRIEF DESCRIPTION OF THE DRAWINGS

FIG. 1 is a three dimensional exploded view of the syringe safety device of the present invention.

FIG. 2 is an exploded view of the combined first buckle seat and the second buckle seat as well as the protective cover of the syringe safety device of the present invention.

FIG. 3 is the first assembled side view of the syringe safety device of the present invention.

FIG. 4 is the second assembled side view of the syringe safety device of the present invention.

FIG. 5 is an outer view of the syringe and the needle cover used together with the first buckle seat and the second buckle seat of the syringe safety device of the present invention.

FIG. 6 is an outer view of the first buckle seat and the second buckle seat of the syringe safety device of the present invention assembled to the syringe and the needle cover.

FIG. 7 is a side view of the syringe safety device of the present invention assembled to the syringe and the needle cover.

FIG. 8 is a side view of the syringe safety device of the present invention assembled to the syringe.

FIG. 9 is a side view of the syringe safety device of the present invention in use for protection and covering of the syringe.

FIG. 10 is a view in the direction of the arrow X shown in FIG. 9.

### DETAILED DESCRIPTION OF THE PREFERRED EMBODIMENTS

In order to provide a better understanding of the structure and functions of the present invention, specific embodiments will be described in detail below. Various different objects disclosed in the embodiments are described roughly in accordance with the relative proportions, dimensions, deformations, or displacements provided in the specification, and are not necessarily drawn according to actual component scale. The directional descriptions made in the present invention such as above, below, left, right, inside, outside etc. are made in accordance with the orientation of the figures, and made in terms of relative directions, instead of absolute directions.

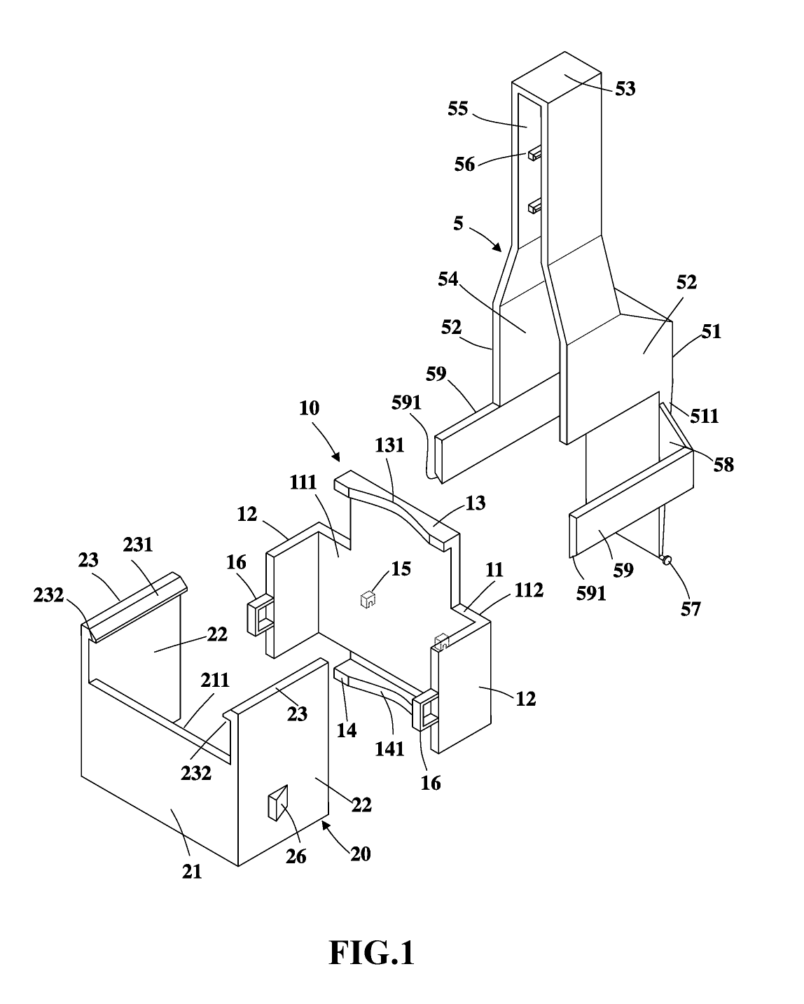

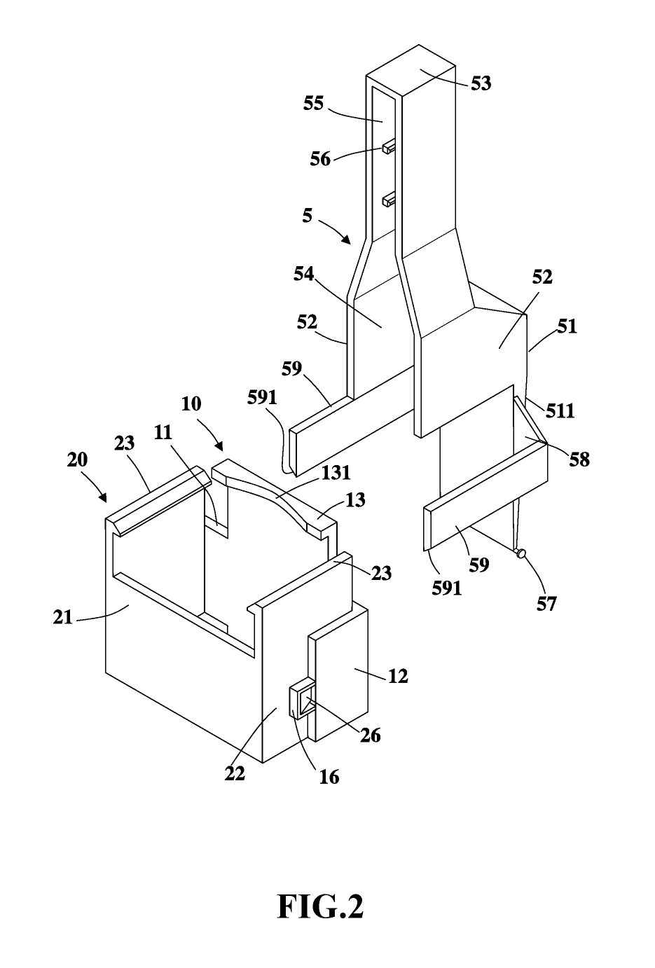

As shown in FIGS. 1 and 2, the protective cover type safety device for syringes according to the present invention comprises a first buckle seat 10 and a second buckle seat 20 which can be combined face to face with each other, as well as a protective cover 5 that can be connected to the first buckle seat 10. The first buckle seat 10, the second buckle seat 20, and the protective cover 5 are all made of plastic material.

The first buckle seat 10 comprises a flat board-shaped positioning portion 11 and wings 12 orthogonally extending from the positioning portion 11 at two sides thereof. Each of the two wings 12 comprises a fastener 16 which is a square fastener with a square hole. The positioning portion 11 comprises flanges 13, 14 that protrude towards the second buckle seat 20 at the upper and lower ends, and the flanges 13, 14 each comprise an arc end surface 131/141. The surface of the positioning portion 11 that faces the second buckle seat 20 is the inner face 111, while the other side surface is the outer face 112. The inner face 111 comprises an anti-slip structure, and the anti-slip structure can be formed of distributed bumps/embossed points or fine ribs. The outer face 112 has a pivoting portion 15, and the pivoting portion 15 is a structure that can receive a round shaft 57 and allow free rotation of the round shaft 57.

The second buckle seat 20 comprises a flat board-shaped positioning portion 21 and wings 22 orthogonally extending from the positioning portion 21 at two sides thereof. Each of the two wings 22 comprises a fastener 26 and the fasteners 26 are protruding pieces that can be combined respectively with the above said fasteners 16 in a way that they cannot be easily separated. The top of the two wings 22 each comprise a limiting member 23 that extends towards each other. The top surfaces of the limiting members 23 each have a guiding portion 231, and the guiding portion 231 is an inclined surface that guides a contacted object towards the inside of the second buckle seat 20. The bottom of the limiting member 23 has a stop portion 232, and the stop portion 232 serves to stop a contacted object from moving upwards and thus from being separated out. The surface of the positioning portion 21 that faces the second buckle seat 10 is the inner face 211. The inner face 211 comprises an anti-slip structure, and the anti-slip structure may be formed of distributed bumps/embossed points or fine ribs.

Initially, the first buckle seat 10 and the second buckle seat 20 are made as two independent portions (See, FIG. 1), but the first buckle seat 10 and the second buckle seat 20 can be connected to each other (see FIG. 2), in which case, the second buckle seat 20 is positioned in between the two wings 12 of the first buckle seat 10. The inner side surfaces of the two wings 12 of the first buckle seat 10 contact the outer side surfaces of the two wings 22 of the second buckle seat 20, where the two fasteners 16, 26 are respectively fastened to each other. A distance is formed between the positioning portion 11 of the first buckle seat 10 and the positioning portion 21 of the second buckle seat 20.

The protective cover 5 comprises a back plate 51, two side plates 52 that are respectively connected to the two sides of the back plate 51, and a top panel 53 that is connected to the top surface of the back plate 51 and the top surfaces of the two side plates 52. The geometrical shapes and dimensions of the back plate 51, side plates 52, and the top panel 53 define two accommodating spaces which are the first accommodating space 54 and the second accommodating space 55 of the protective cover 5. The second accommodating space 55 is narrower than the first accommodating space 54. The two side plates 52 comprise protruding clamping ribs 56 on the inner side surface of the second accommodating space 55. The back plate 51 comprises a round shaft 57 at the bottom thereof. The two side plates 52 comprise two extension arms 58 respectively extending towards the left and the right sides. The distal end of each one of the two extension arms 58 also comprises a hugging arm 59, and the two hugging arms 59 extend parallel to each other. The outer distance between the two hugging arms 59 (the distance between their respective outer side surfaces) is equal to the inner distance between the two wings 22 of the second buckle seat 20 (the distance between its respective inner side surfaces). The outer side surfaces of the two hugging arms 59 both comprise a bevel face 591 at the bottom thereof. The back plate 51 comprises a pushing surface 511 at a position near the round shaft 57.

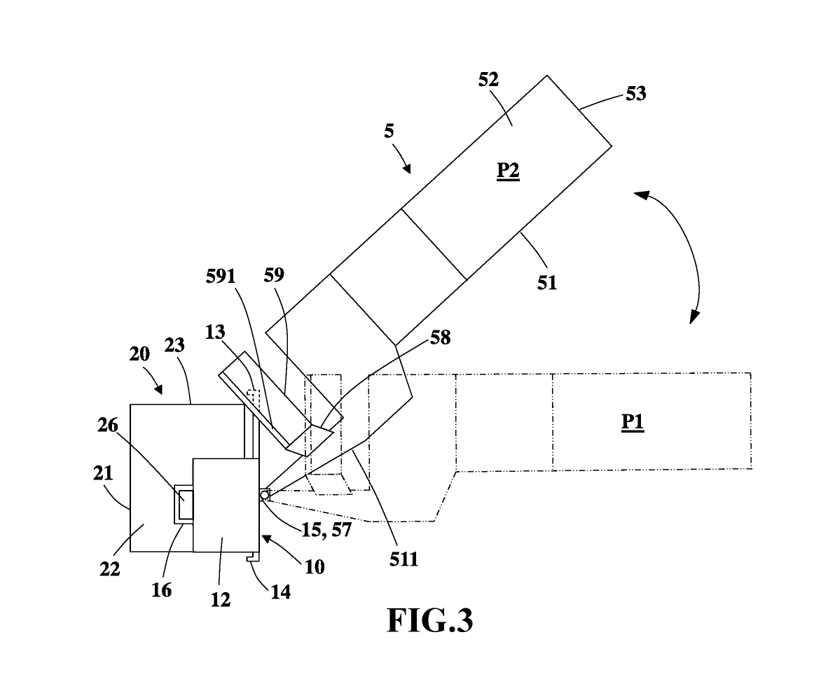

As shown in FIG. 3, the protective cover 5 is connected to the pivoting portion 15 of the first buckle seat 10 via the round shaft 57. The protective cover 5 can be circularly swung about the round shaft 57 between a first position P1 and a second position P2.

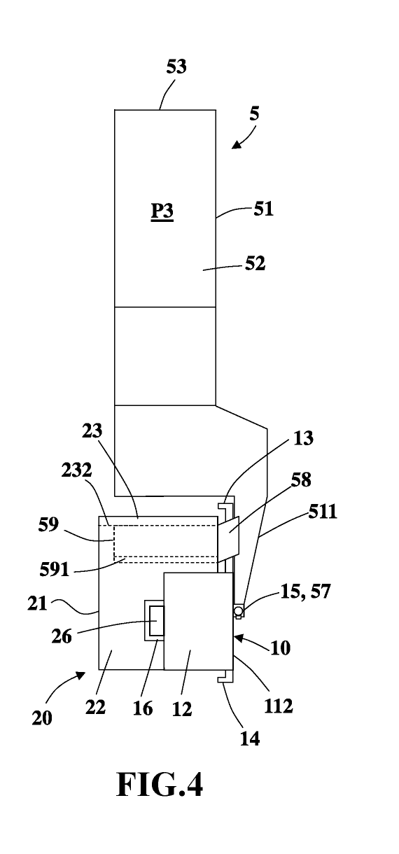

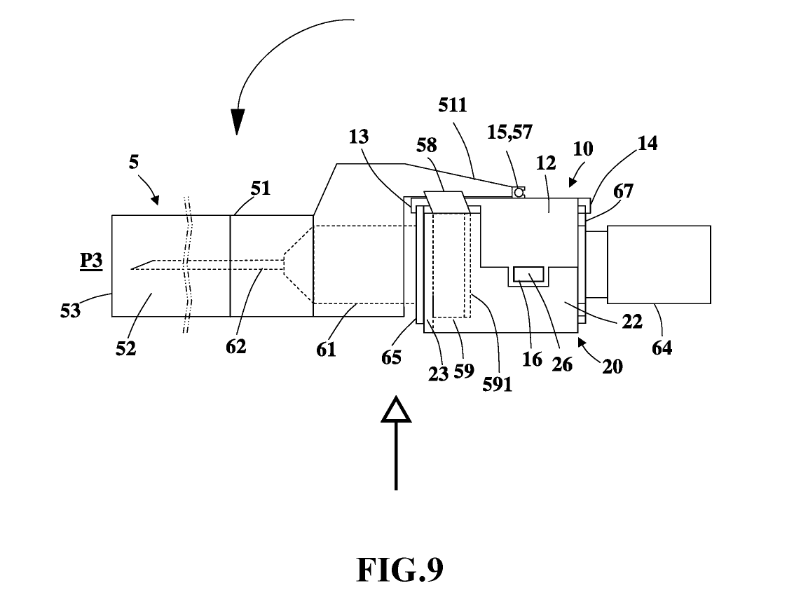

As shown in FIG. 4, when an external pushing force is applied on the protective cover 5, the protective cover 5 is moved from the first position P1 (or the second position P2) into a third position P3 and is engaged at the third position P3. In other words, by means of a pushing force applied on the pushing surface 511 of the protective cover 5, the protective cover 5 is circularly swung about the round shaft 57 from the first position P1 (or the second position P2) further on towards the direction of the first buckle seat 10 and the second buckle seat 20. As the hugging arms 59 are pushed downward by the pushing force, the guiding portion 231 of the limiting member 23 come in contact with the bevel faces 591 of the hugging arms 59, and the hugging arms 59 are pushed against the limiting members 23, then the hugging arms 59 bend inward slightly, and the limiting members 23 bend outward slightly. Therefore the hugging arms 59 can pass the limiting members 23 and reach underneath the limiting members 23, and the protective cover 5 enters into the third position P3. As shown in FIG. 9, when the hugging arms 59 are engaged under the limiting members 23, due to the obstruction of the stop portions 232, the hugging arms 59 cannot move back and separate from the second buckle seat 20, and the protective cover 5 can no longer swing anymore. The protective cover 5 cannot move from the third position P3 back to the first position P1 or the second position P2.

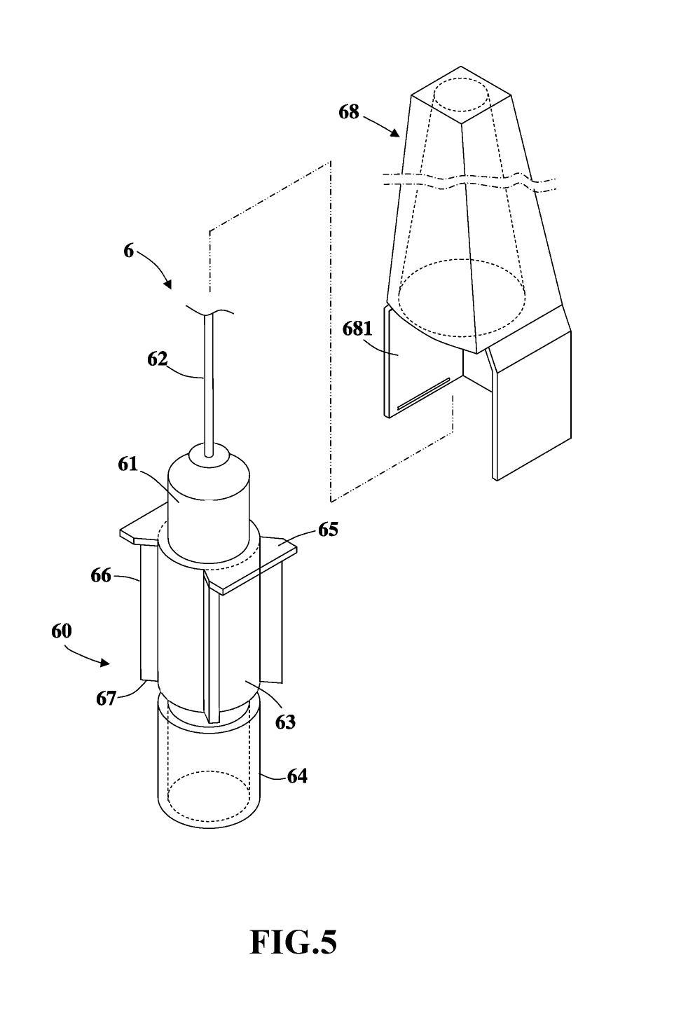

As shown in FIG. 5, the syringe 6 is a medically used intravenous catheter already known in the prior art. The syringe 6 comprises a needle seat 60, and the needle seat comprises a fixed portion 61 to be fixedly connected with a needle body 62, as well as a pipe joint part 63 to be connected to a pipe casing 64. The pipe casing 64 is fitted with an infusion tube (now shown) for intravenous injection. Intravenous injection flows out from the needle body 62 via the pipe joint part 63. A platform 65 with a large external diameter is connected between the fixed part 61 and the pipe joint part 63. The outer walls of the pipe joint part 63 comprise several protruding ribs 66 that are parallel to the axial direction of the pipe joint part 63. One end of the protruding ribs 66 is connected to the platform 65, while the other end of the protruding ribs 66 extends to the distal end of the pipe joint part 63, and thus the distal end of the pipe joint part 63 comprises several latching portions 67 that radially protrude outward. A long cylindrical plastic needle cover 68 is mounted to the outer portion of the platform 65, and covers the fixed part 61 and the needle body 62. The needle cover 68 comprises an opening 681.

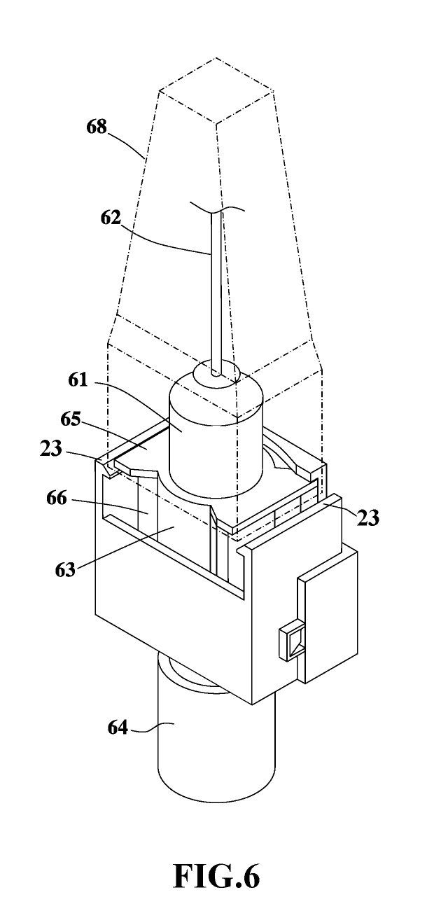

As shown in FIGS. 5 and 6, the first buckle seat 10 and the second buckle seat 20 are two individual parts. They both face the pipe joint part 63, and are combined as shown in FIG. 2. The pipe joint part 63 is tightly clamped between the first buckle seat 10 and the second buckle seat 20. By means of the anti-slip structure at the inner faces 111 of the first buckle seat 10 and the inner face 211 of the second buckle seat 20, the first buckle seat 10 and the second buckle seat 20 are force-fitted to the pipe joint part 63. In addition, the flange 13 at the top end of the first buckle seat 10 contacts and restrict the platform 65 via the opening 681 of the needle cover 68. The flange 14 at the bottom of the first buckle seat 10 is restricted beneath the latching portions 67 of the pipe joint part 63, such that no upward and downward movement of the first buckle seat 10 and the second buckle seat 20 relative to the pipe joint part 63 is allowed. The first buckle seat 10 and the second buckle seat 20 are tightly mounted around the pipe joint part 63 of the needle seat 60. The limiting members 23 of the second buckle seat 20 are located below the platform 65. As shown in FIG.. 7, the protective cover 5 can swing freely between the first position P1 and the second position P2. Preferably, at the second position P2, the protective cover 5 holds the plastic needle cover 68 by the two hugging arms 59. The friction between the plastic hugging arms 59 and the plastic needle cover 68 is sufficient to restrict the protective cover 5 from easily overturning outward and getting separated from the needle cover 68.

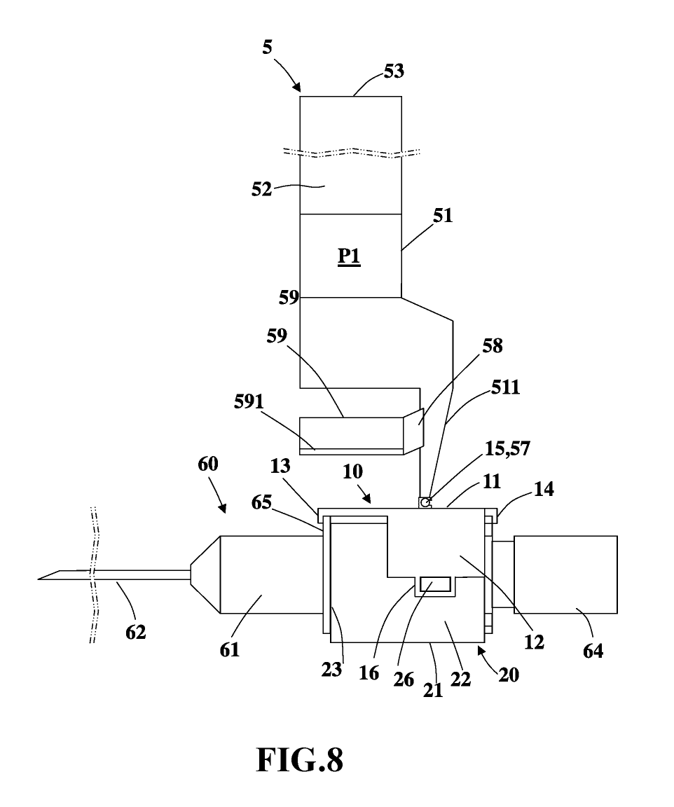

As shown in FIG. 8, when injection is to be made, by applying a light force, the hugging arm 59 of the protective cover 5 are separated from the needle cover 68, and then the protective cover 5 is set at the first position P1, and the needle cover 68 is dismounted, so that the injection can be made with the needle body 62. Since the first buckle seat 10 and the second buckle seat 20 are planar structures, medical personnel can carry out injection while stably holding the first buckle seat 10 and the second buckle seat 20. Besides, the protective cover 5 at the first position does not interrupt the injection operation.

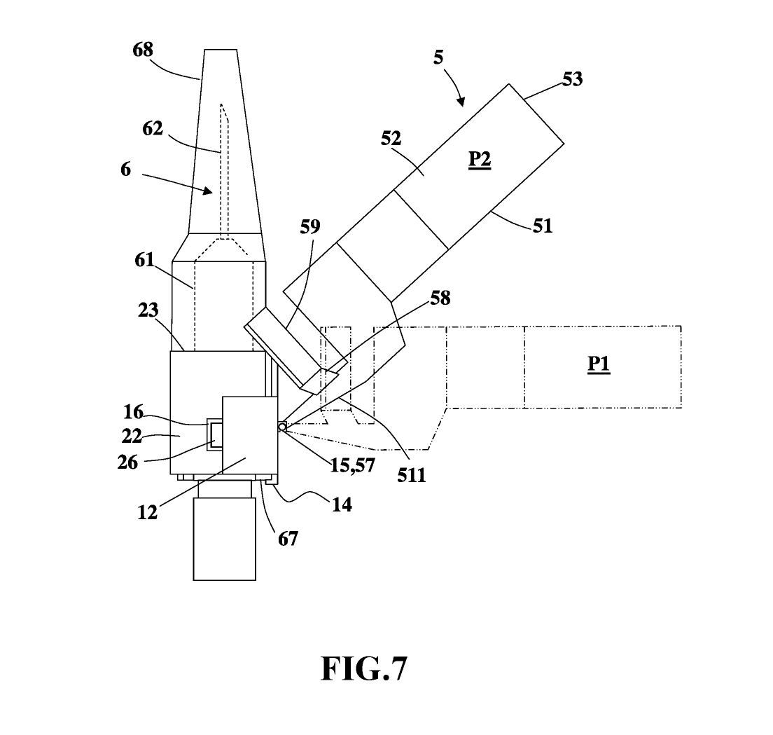

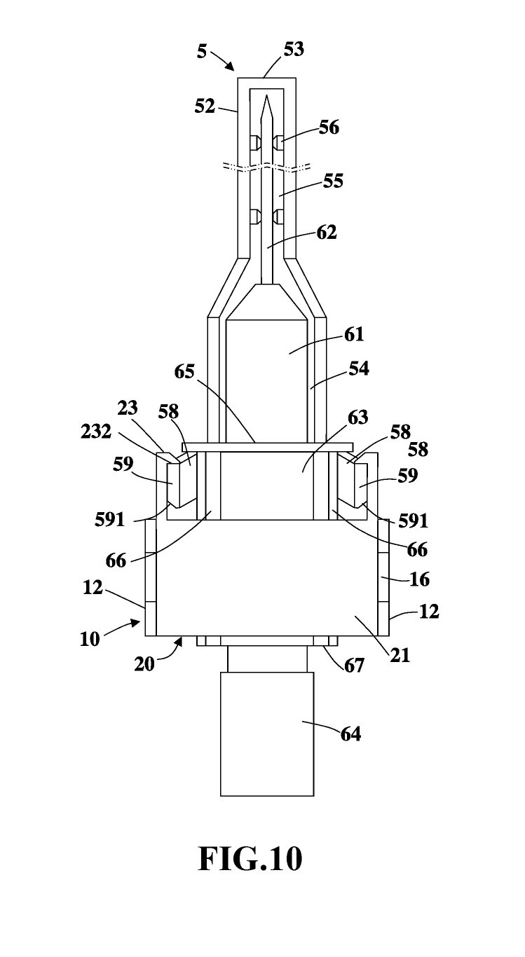

As shown in FIGS. 9 and 10, after use, the user's forefinger that holds the needle device applies a pressure to the pushing surface 511 to push the protective cover 5 towards the needle body 62. The hugging arms 59 are pushed by the pressure and guided by the bevel faces 591 and the guiding portions 231, the hugging arms 59 are guided towards underneath the limiting members 23 of the second buckle seat 20. The protective cover 5 is connected to the first buckle seat 10 in an upright position, and the platform 65 and the fixed part 61 are accommodated in the first accommodating space 54, and the needle body 62 is accommodated in the second accommodating space 55. The side plates 52 clamp and hold the needle body 62 via the clamping ribs 56. At this point, the protective cover 5 completely covers and protects the needle body 62 after use. Also, as shown in FIG. 7 and disclosed above, when the hugging arms 59 are stopped by the stop portions 232, the hugging arms 59 cannot move from the third position P3 back to the first position P1 or the second position P2, and the needle body 62 is covered by the protective cover 5 and is not exposed.

In the above given figures and embodiments, the present invention is able to fit with an already known intravenous catheter, but it is not restricted to this kind of syringes. The present invention can be adapted to other types of syringes such as butterfly syringes, attached syringes, etc. simply by making the inner faces 111 of the positioning portions 11, the inner face 211 of the positioning portion 21 of the present invention into a shape that accommodates the cross sectional shape of the needle seat of the conventional syringes.

What is claimed is:

1. A protective cover type safety device for a syringe that has a needle seat, a needle body connected to the needle seat and a needle cover covering the needle body, the safety device comprising:
a first buckle seat having a positioning portion and two wings respectively coupled to two sides of the positioning portion, each of the two wings having a fastener, a pivoting portion being formed on an outer side of the positioning portion;
a second buckle seat having a positioning portion and two wings respectively coupled to two sides of the positioning portion of the second buckle seat, each of the two wings of the second buckle seat having a fastener, a limiting member extending from a top thereof, the fastener of the second buckle seat being connectable to the fastener of the first buckle seat to clamp and position the first buckle seat and the second buckle seat to an exterior of the needle seat of the syringe; and
a protective cover having a back plate, two side plates respectively coupled to two sides of the back plate, a top plate coupled to a top of the back plate and a top of each the two side plates, a first accommodating space and a second accommodating space being defined between the back plate, the side plates and the top plate, the first accommodating space communicating with the second accommodating space, a bottom edge of the back plate having a round shaft, two extension arms respectively extending from the two side plates, a hugging arm extending from an end of each of the two extension arms, the two hugging arms being parallel to each other;
wherein the protective cover is connected to the pivoting portion of the first buckle seat by the round shaft and capable of swinging about the round shaft between a first position and a second position, the protective cover may be pushed from the first position by an external pushing force to be engaged in a third position and thus unable to return to the first position;
the protective cover embraces the needle cover of the syringe by the two hugging arms when the protective cover is swung to the second position;
when the protective cover is swung outwardly from the second position to the first position, the protective cover is offset from the needle cover of the syringe, so that the needle cover may be removed from the syringe, and the needle body is ready for injection; and
when the needle body is used, an external pushing force may be applied to the protective cover to swing the protective cover from the first position to the third position, so that the protective cover is latched below the limiting members by passing the two hugging arms by the limiting members, and the protective cover accommodates the needle body and the needle seat of the syringe in the first accommodating space and the second accommodating space.

2. The protective cover type safety device for a syringe according to claim 1, wherein the fastener of the first buckle seat is a square fastener with a square hole and the fastener of the second buckle seat is a protruding member which is capable of engaging with the fasteners.

3. The protective cover type safety device for a syringe according to claim 1, further comprising a flange extending from an upper end of the positioning portion of the first buckle seat, wherein when the needle seat has a platform with an expanded diameter and located at a position above the first buckle seat and the second buckle seat, the flange is stopped at the platform.

4. The protective cover type safety device for a syringe according to claim 3, further comprising a flange extending from at a lower end of the positioning portion of the first buckle seat, wherein when a plurality of ribs are formed on an outer wall of the needle seat and extending in parallel to an axial direction of the needle seat to form a plurality of outwardly protruding latching portions at a bottom edge of the needle seat, the flange at the lower end of the first buckle seat is stopped at the latching portions.

5. The protective cover type safety device for a syringe according to claim 4, wherein the flanges at the upper and lower ends of the positioning portion of the first buckle seat each have an arc end surface.

6. The protective cover type safety device for a syringe according to claim 1, wherein an inner surface of the positioning portion of the first buckle seat and an opposite inner surface of the positioning portion of the second buckle seat each comprises an anti-slip structure.

7. The protective cover type safety device for a syringe according to claim 1, wherein the protective cover has a bevel face disposed at a bottom of each of the two hugging arms, the second buckle seat has a guiding portion formed at a top of each of the two limiting members for guiding the bevel face to move downward, so that the hugging arms are guided below the limiting members and stopped by stop portions formed at a bottom of the limiting members to prevent the hugging arms from separating in an opposite direction.

8. The protective cover type safety device for syringes according to claim 1, wherein the two side plates of the protective cover each have a protruding clamping rib disposed thereon and configured to be opposite to the inner surface of the second accommodating space.

9. The protective cover type safety device for syringes according to claim 1, wherein the back plate of the protective cover has a pushing surface.
